(19) United States
(12) Patent Application Publication
(10) Pub. No.: US 2022/0082549 A1
(43) Pub. Date: Mar. 17, 2022

(54) INTEGRATED ELECTROPHYSIOLOGY AMPLIFYING APPARATUS, COMPUTER-ACCESSIBLE MEDIUM, SYSTEM AND METHOD FOR USE THEREOF

(71) Applicant: The Trustees of Columbia University in the City of New York, New York, NY (US)

(72) Inventors: Siddharth Shekar, Santa Clara, CA (US); Krishna Jayant, West Lafayette, IN (US); Kenneth L. Shepard, Ossining, NY (US)

(21) Appl. No.: 17/534,903

(22) Filed: Nov. 24, 2021

Related U.S. Application Data
(63) Continuation of application No. PCT/US2020/034540, filed on May 26, 2020.
(60) Provisional application No. 62/852,587, filed on May 24, 2019.

(51) Int. Cl. G01N 33/487; B01L 3/02

(52) U.S. Cl. CPC G01N 33/48728; B01L 3/0237

(57) ABSTRACT

Exemplary embodiments of the present invention provide for an integrated electrophysiology amplifying apparatus, computer-accessible medium, system and method for use thereof. In accordance with certain exemplary embodiments of the present disclosure, an integrated electrophysiology amplifying system can include: a pipette interface for receiving a pipette or sharp microelectrode; and an integrated circuit having (i) an amplifier coupled to the pipette interface and configured to control a current through a connected pipette or record a cell membrane voltage and (ii) at least one compensation circuit using negative feedback; wherein the integrated circuit and pipette interface are physically integrated within a common housing.

### CROSS-REFERENCE TO RELATED APPLICATION(S)

This application relates to and claims priority from U.S. Patent Application Ser. No. 62/852,587, filed on May 24, 2019, the entire disclosure of which is incorporated by herein by reference.

### STATEMENT REGARDING FEDERALLY SPONSORED RESEARCH

This invention was made with government support under Grant Nos. 01NS099717 and U01NS099697, awarded by the National Institutes of Health (NIH), Grant No. N66001-17-C-4002, awarded by the Defense Advanced Research Projects Agency (DARPA), as well as Contract No. W911NF-12-1-0594 (MURI), awarded by the United States Army Research Office. The government has certain rights in the invention.

### FIELD OF THE DISCLOSURE

The present disclosure relates generally to electrophysiology, and more specifically, to exemplary embodiments of an exemplary integrated electrophysiology amplifying apparatus, computer-accessible medium, system and method for use thereof

### BACKGROUND INFORMATION

Intracellular electrophysiological recordings from neurons are a high-fidelity neuroscience procedure that enable fundamental understanding of neuronal computation and function. These recordings are typically performed using electrolyte-filled glass pipettes in either whole-cell or sharp electrode configurations. Pipettes used in the whole-cell configuration typically have diameters on the order of a few μm and impedances on the order of a few MΩ. In this configuration, the pipette tip is positioned close to the cell such that it first forms a loose seal with the membrane—commonly referred to as the “cell-attached” configuration. Upon subsequent application of suction, the tip-membrane interface forms a giga-seal, and any further increase in the suction ruptures the membrane yielding full intracellular access. The whole-cell procedure is the current gold-standard and results in precise measurement of intracellular currents and voltages. Alternatively, sharp electrodes have diameters on the scale of a few nm and impedances on the order of 100 MΩ and are used to impale the cell membrane to gain intracellular access for accurate voltage measurements. An amplifier connected to the pipette can be used to control the current through the pipette and record the membrane voltage (current-clamp, CC) or control the voltage in the membrane and record the membrane current (voltage-clamp, VC). CC facilitates the measuring of the voltage response of a cell to electrochemical stimuli. VC, on the other hand, can be used to determine the composition and concentration of voltage sensitive ion channels in the membrane, which can have significant implications, for example, in drug discovery.

Recording these μV-to-mV-scale voltages and pA-to-nA-scale currents necessitates the use of precision low-noise instrumentation amplifiers. The recordings are further complicated by the series resistance (Rₛ) and capacitance (Cₚ) of the pipette which, in the best case, distort the recordings and, in the worst case, lead to a complete loss of clamping ability. The amplifier preferably has associated compensation circuitry to account for these non-idealities in the pipette. Benchtop amplifiers, such as the Axopatch 200B and the Axopatch 700B, perform these recordings with high signal-to-noise ratio (SNR). However, they use discrete components in their design, increasing the cost, weight, and associated wiring parasitics of these systems which consequently limits their bandwidth, scalability, power efficiency, and performance.

Thus, it may be beneficial to provide an exemplary integrated electrophysiology amplifying apparatus, computer-accessible medium, system and method for use thereof which can overcome at least some of the deficiencies described herein above.

### SUMMARY OF EXEMPLARY EMBODIMENTS

An exemplary integrated electrophysiology amplifying apparatus, computer-accessible medium, system and method can be provided which can include and/or utilize a pipette interface for receiving a pipette or sharp microelectrode, and an integrated circuit having (i) an amplifier coupled to the pipette interface and configured to control a current through a connected pipette or record a cell membrane voltage, and (ii) at least one compensation circuit using negative feedback. The integrated circuit and pipette interface can be physically integrated within a common housing.

Another exemplary integrated electrophysiology amplifying apparatus, computer-accessible medium, system and method be provided which can include and/or utilize, e.g., a pipette interface for receiving a pipette or sharp microelectrode; and an integrated circuit having (i) an amplifier coupled to the pipette interface and configured to control a cell membrane voltage or record a trans-membrane current, and (ii) at least one compensation circuit using negative feedback. The integrated circuit and pipette interface can be physically integrated within a common housing.

In some exemplary embodiments of the present disclosure, the amplifier can include a current-clamp module to control the current through the pipette, and a voltage-clamp module to control the cell membrane voltage. The current-clamp and voltage-clamp modules can share an input. Further, at least one compensation circuit can compensate a series resistance associated with the pipette. For example, at least one compensation circuit can compensate for the series resistance over a range greater than 100 MΩ. The amount of series resistance compensated can be programmed via a digital interface. Further, at least one compensation circuit can compensate for a capacitance associated with the pipette. For example, at least one compensation circuit can compensate for the capacitance associated with the pipette over a range greater than 10 pf.

These and other objects, features and advantages of the exemplary embodiments of the present disclosure will become apparent upon reading the following detailed description of the exemplary embodiments of the present disclosure, when taken in conjunction with the appended claims.

### BRIEF DESCRIPTION OF THE DRAWINGS

Further objects, features and advantages of the present disclosure will become apparent from the following detailed description taken in conjunction with the accompanying Figures showing illustrative embodiments of the present disclosure, in which:

FIG. 1A illustrates an exemplary simplified block diagram for neuronal intracellular recordings in accordance with certain exemplary embodiments of the present disclosure;

FIG. 1B illustrates an exemplary schematic diagram of a current-clamp showing the implementation of a voltage buffer, Cₚ compensation circuitry, and current injection circuitry in accordance with certain exemplary embodiments of the present disclosure;

FIG. 1C illustrates an exemplary transistor-level schematic diagram of a rail-to-rail input, rail-to-rail output operational transconductance amplifier in accordance with certain exemplary embodiments of the present disclosure;

FIG. 1D illustrates an exemplary schematic diagram of a voltage-clamp showing the implementation of a transimpedance amplifier (TIA), Cₚ compensation circuitry, and Rₛ compensation circuitry in accordance with certain exemplary embodiments of the present disclosure;

FIG. 1E illustrates an exemplary diagram of the amplifier integrated circuit die in accordance with certain exemplary embodiments of the present disclosure;

FIG. 1F illustrates an exemplary image of the printed circuit board including the integrated circuit in accordance with certain exemplary embodiments of the present disclosure;

FIG. 1G illustrates an exemplary image of a patch pipette contacting a neuron, as seen from a microscope, in accordance with certain exemplary embodiments of the present disclosure;

FIG. 1H illustrates an exemplary image of a measurement setup consisting of a microscope, manipulator, and an amplifier in accordance with certain exemplary embodiments of the present disclosure;

FIG. 2A illustrates an exemplary concatenated time trace in accordance with certain exemplary embodiments of the present disclosure;

FIG. 2B illustrates a graph of an exemplary power spectral density (PSD) in accordance with certain exemplary embodiments of the present disclosure;

FIG. 2C illustrates a graph of voltage recorded by a buffer (with and without capacitance compensation) in accordance with certain exemplary embodiments of the present disclosure;

FIG. 2D illustrates an exemplary concatenated time trace in accordance with certain exemplary embodiments of the present disclosure;

FIG. 2E illustrates a graph comparing an exemplary PSD in accordance with certain exemplary embodiments of the present disclosure with a known system;

FIG. 2F illustrates a graph of current recorded by a TIA (with and without capacitance compensation) in accordance with certain exemplary embodiments of the present disclosure;

FIG. 2G illustrates a graph of current recorded by a TIA (with and without resistance compensation) in accordance with certain exemplary embodiments of the present disclosure;

FIG. 3A illustrates an example of an in vitro recording using sharp microelectrodes in accordance with certain exemplary embodiments of the present disclosure;

FIG. 3B illustrates an example of extracellular action potentials recorded using sharp microelectrodes in accordance with certain exemplary embodiments of the present disclosure;

FIG. 3C illustrates an example of intracellular action potentials recorded using sharp microelectrodes in accordance with certain exemplary embodiments of the present disclosure;

FIG. 3D illustrates an example of a spike-triggered average of eleven action potentials recorded using a MultiClamp 700B;

FIG. 4A illustrates an example of an in vitro recording using patch pipettes in accordance with certain exemplary embodiments of the present disclosure;

FIG. 4B illustrates an example of a loose-seal VC recording from a neuron using a patch pipette in accordance with certain exemplary embodiments of the present disclosure;

FIG. 4C illustrates an example of a tight-seal VC recording from a neuron using a patch pipette in accordance with certain exemplary embodiments of the present disclosure;

FIG. 4D illustrates an example of excitatory and inhibitory postsynaptic potentials in accordance with certain exemplary embodiments of the present disclosure;

FIG. 5A illustrates an exemplary schematic diagram of a capacitance compensation circuit in accordance with certain exemplary embodiments of the present disclosure;

FIG. 5B illustrates an exemplary block diagram of the resistance compensation circuit in accordance with certain exemplary embodiments of the present disclosure;

FIG. 6A illustrates exemplary Bode plots associated with the resistance compensation circuit in accordance with certain exemplary embodiments of the present disclosure;

FIG. 6B illustrates an exemplary Bode plot associated with the resistance compensation circuit in accordance with certain exemplary embodiments of the present disclosure;

FIG. 6C illustrates a graph of a current recorded by the TIA in accordance with certain exemplary embodiments of the present disclosure;

FIG. 6D illustrates an example of a voltage output for a current clamp implementing capacitance compensation in accordance with certain exemplary embodiments of the present disclosure;

FIG. 6E illustrates an example of a frequency response for a voltage clamp TIA in accordance with certain exemplary embodiments of the present disclosure;

FIG. 6F illustrates a graph of a voltage clamp's TIA linearity as a function of input amplitude before adjustment in accordance with certain exemplary embodiments of the present disclosure;

FIG. 6G illustrates a graph of the voltage clamp's TIA linearity as a function of input amplitude before adjustment in accordance with certain exemplary embodiments of the present disclosure; and

FIG. 7 illustrates an exemplary block diagram of an exemplary system in accordance with certain exemplary embodiments of the present disclosure.

Throughout the drawings, the same reference numerals and characters, unless otherwise stated, are used to denote like features, elements, components or portions of the illustrated embodiments. Moreover, while the present disclosure will now be described in detail with reference to the figures, it is done so in connection with the illustrative embodiments and is not limited by the particular embodiments illustrated in the figures and the appended claims.

### DETAILED DESCRIPTION OF EXEMPLARY EMBODIMENTS

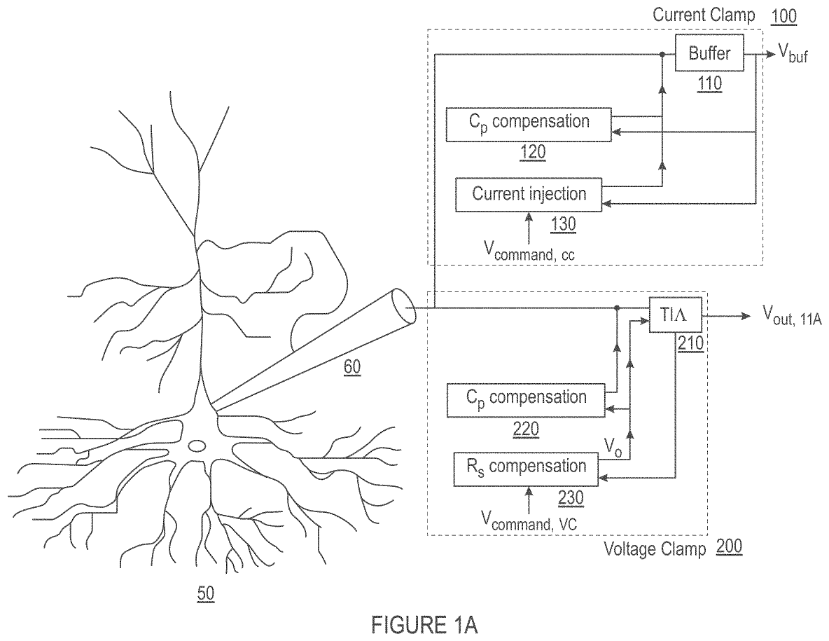

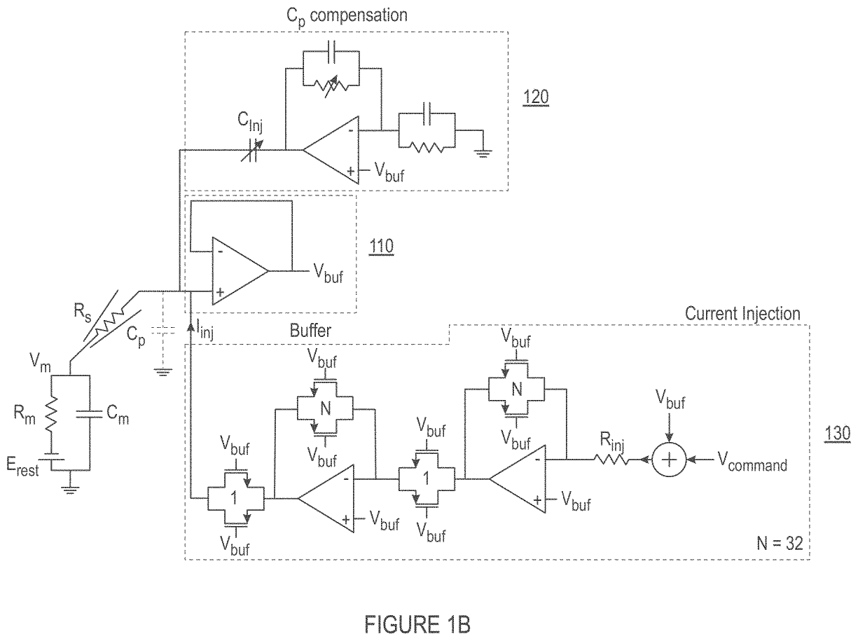

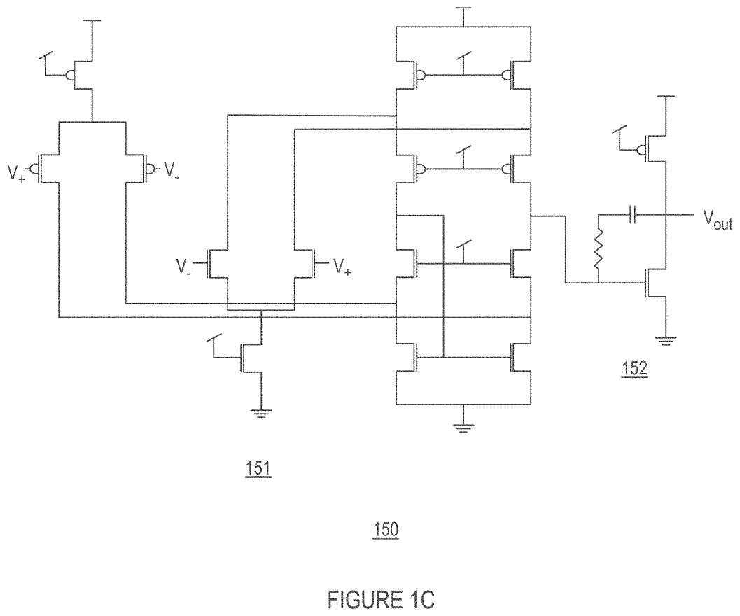

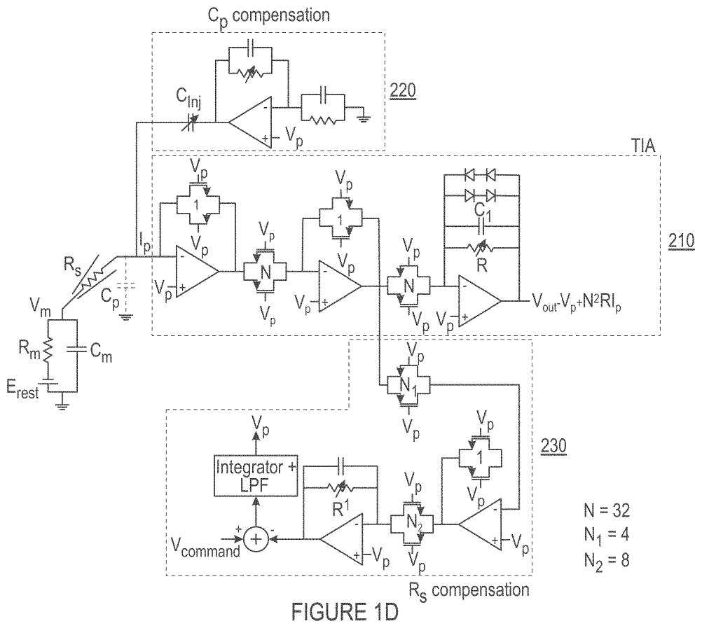

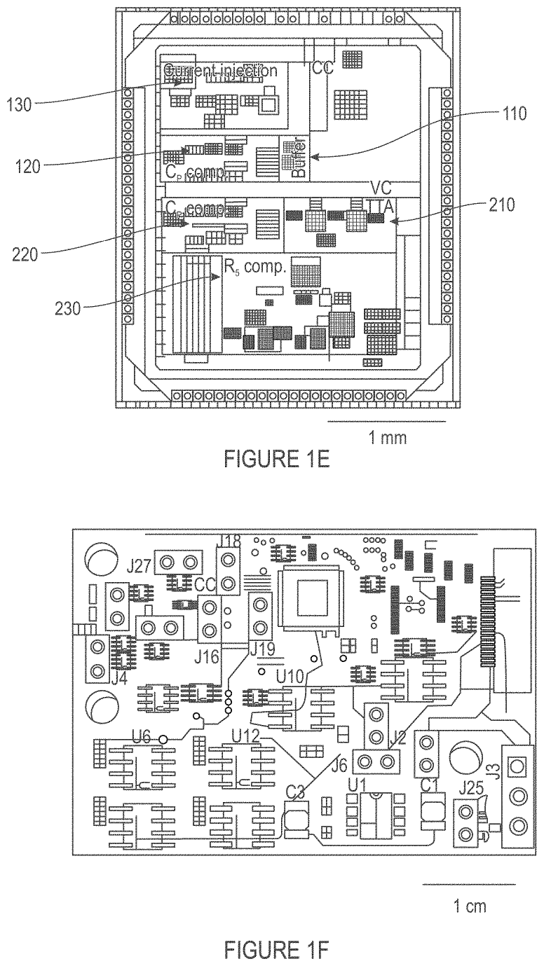

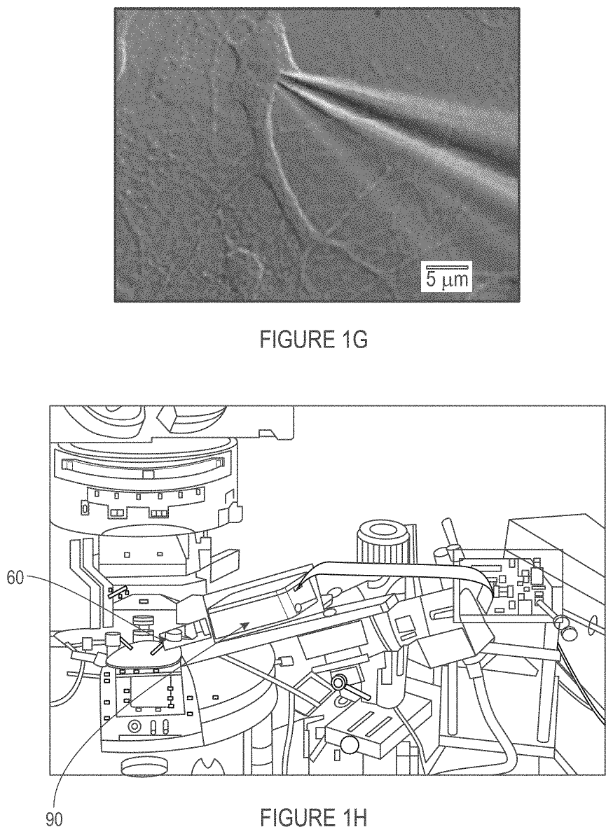

FIG. 1A illustrates an exemplary schematic diagram for neuronal intracellular recordings in accordance with certain exemplary embodiments of the present disclosure. In particular, the figure illustrates an experiment for recording intracellular signals from a neuron 50 and a block diagram of the associated electronics. In accordance with certain exemplary embodiments of the present invention, a current clamp 100 can consist of a voltage buffer 110 with a high-impedance input and unity gain. In the special case where the current being injected in (Iᵢₙⱼ) is zero, the only extra circuitry required is that needed to compensate for Cₚ, which acts in conjunction with Rₛ to filter the measured signal. Cₚ can be typically of the order of a few pF and is governed by the geometry and insertion depth of a pipette 60. However, Iᵢₙⱼ is generally not zero and, therefore, it is beneficial to program Iᵢₙⱼ in the pA—nA range. The programmability can be achieved by varying an externally applied command voltage (V_{command}). When a current is being injected, Rₛ introduces a proportional offset voltage in the measurement. If Rₛ is determined accurately prior to the experiment and is assumed to remain unchanged, this offset is easily subtracted. In the absence of Rₛ, a voltage clamp 200 can be achieved using a current-to-voltage converter, also known as a transimpedance amplifier (TIA) 210, that ensures that the voltage (Vₚ) of the pipette 60 equals to V_{command}. However, Rₛ can typically be several 10's of MΩ for patch pipettes and can be several hundreds of MΩ for sharp microelectrodes. Signal currents, which are typically in the pA—nA range, flow through this Rₛ and can cause mV-scale errors in the clamp voltage. In addition, Rₛ in combination with the membrane capacitance, Cₘ, filters the signal of interest. Hence, dedicated circuits are preferably used to compensate for Rₛ. Cₚ compensation, which can include Cₚ circuitry 120, 220, may be beneficial in order to accurately determine the signal current, which may be beneficial for stable Rₛ compensation 230 which can include Rₛ circuitry 230.

FIG. 1B illustrates an exemplary schematic diagram of a current-clamp 100 showing the implementation of a voltage buffer 110, Cₚ compensation circuitry 120, and current injection circuitry 130 in accordance with certain exemplary embodiments of the present disclosure. The voltage buffer 110 with unity gain can be implemented as an operational amplifier (op-amp) in negative feedback. The buffer 110 can beneficially have low input leakage current so as to facilitate for voltage recordings with Iᵢₙⱼ=0. Hence, the op-amp can be designed with thick-oxide metal-oxide-semiconductor field effect transistor (MOSFET) inputs to ensure that the input leakage current is <10 fA.

FIG. 1C illustrates an exemplary transistor-level schematic diagram of a rail-to-rail input, rail-to-rail output operational transconductance amplifier 150 in accordance with certain exemplary embodiments of the present disclosure. As depicted in FIG. 1C, the first stage can consist of a dual n- and p-input folded cascode 151 followed by a common-source second stage 152. The dual inputs enable rail-to-rail input swing while the common-source second stage 152 facilitates rail-to-rail output swing. The preferred and/or required bias voltages can be generated in a separate biasing block.

Cₚ compensation can be employed for CC in order to measure voltage signals at the highest possible bandwidth. For a voltage signal Vₘ generated in the cell membrane, in the absence of Cₚ compensation, the voltage recorded by the buffer can be Vₘ filtered by Rₛ and Cₚ. For example, using the patch pipette 60 with Rₛ=25 MΩ and Cₚ=5 pF, this can set the 3-dB bandwidth for the recording at 1.27 kHz. For sharp microelectrodes with higher Rₛ and Cₚ, this gets proportionately worse. Cₚ compensation can be achieved by multiplying the recorded voltage V_{buf} by a programmable factor A (1<A<2) and connecting this back to the input through a programmable capacitor Cᵢₙⱼ. The current injected back in is

$$( A - 1 ) {C}_{inj} \frac{{d V}_{buf}}{dt} .$$

In one embodiment, A can be implemented using an op-amp as a non-inverting amplifier with programmable feedback resistance that gives 10-bits of resolution. Cᵢₙⱼ can be selectable, such as between 0, 5, 10 and 15 pF. The compensation step-size of Cᵢₙⱼ/1024 depends on the value of Cᵢₙⱼ selected and is typically less than 5 fF when the 5 pF capacitor is selected.

Current injection can be used as a stimulus to characterize the voltage response of the cell. Considering that the membrane resistance of the cell, Rₘ, can be several 10's of MΩ or larger, the output impedance of the current injection block/circuitry 130 needs to beneficially be at least an order of magnitude larger than this so as to not add substantial amount of leakage current. The current injection block/circuitry 130 can be implemented using, e.g., transistors in the subthreshold regime as active current dividers which is shown in FIG. 1B. The external V_{command} can be first converted into a proportional current through a fixed on-chip resistor Rᵢₙⱼ (e.g., nominally 100 kΩ). This current can then be passed through two stages of 32× current division to yield a net transconductance of 1024×100 kΩ≈100 MΩ. In a further example, ratioed capacitors in parallel with the subthreshold transistors (not shown in figure) can be used to extend the operating bandwidth of the current injection. A large value for the effective injection resistance can be desirable in order to reduce its current noise contribution but places limits on the largest current that can be injected. The active current division can serve to decrease the thermal noise of Rᵢₙⱼ by a factor of N² such that the input-referred noise contribution of Rᵢₙⱼ is then equivalent to that of a passive resistor of value ≈100 GΩ.

FIG. 1D illustrates an exemplary schematic diagram of a voltage clamp200 showing the exemplary implementation of TIA 210, the Cₚ compensation circuitry 220, and the Rₛ compensation circuitry 230 in accordance with certain exemplary embodiments of the present disclosure. TIA 210 can achieve current amplification using similar principles to those employed in the CC current injection block 130 for current division. After two such stages provide a net current amplification of 1024×, the current can be linearly converted into a proportional output voltage using a transimpedance stage with resistive feedback. Further, in accordance with certain exemplary embodiments of the present disclosure, while the current-to-voltage conversion in each of the current-amplifying stages can be non-linear, the op-amp can ensure that corresponding sets of unit-sized transistors experience substantially the same gate-source and drain-source voltages such that the ratio of their currents is primarily determined by the ratio of the number of devices connected between the output and the following stage to the number of devices in feedback around the op-amp, which can be 32 in each of the stages in this design. Further, additional ratio-ed capacitors in parallel with the subthreshold transistors (not shown in the figures) can be used to extend the amplification bandwidth and ensure closed-loop stability for the op-amps. The feedback resistance in an output stage of TIA 210 can be four-bit programmable from 0-225 kΩ. For large transient input currents, the output voltage of a TIA with a fixed value of feedback resistance can saturate and could lead to temporary loss of feedback. Anti-parallel diodes can be used in parallel with the transimpedance resistor (R) in order to ensure that closed-loop feedback is maintained even for large input currents at the expense of limiting the linear range of TIA 210. The effective transimpedance gain of TIA 210 can be R_{f}=N²R. In accordance with certain exemplary embodiments of the present disclosure, the diode's non-linear I-V relationship can be inverted to extend the dynamic range of TIA 210.

Another exemplary advantage of TIA 210 of the present disclosure is a large operating bandwidth. Traditional TIAs implemented with a large passive resistor as the feedback element are limited in bandwidth by the capacitor required in parallel with the resistor to ensure stability. For example, a 100 MΩ resistor in parallel with a 1 pF feedback capacitor limits TIA 210 bandwidth to 1.6 kHz. In contrast, the feedback capacitor in the feedback path of the transimpedance stage exemplified in the present disclosure appears across R. Since this is 1024× smaller than the effective value of the feedback resistance, the corresponding improvement in bandwidth is 1024×. For example, this resistor can be set to 100 kΩ in order to realize R_{f}≈100 MΩ yielding a cutoff frequency of 1.6 MHz.

Cₚ compensation in VC can be beneficial for performing Rₛ compensation. In a typical VC experiment, Vₚ is stepped from its initial value at the resting membrane potential to a different value. Since TIA 210 can ensure that this step is also applied at the electrode connected to the pipette 60, the resultant current measured by TIA 210 can be a combination of the desired current through the pipette 60 and the charging current required for changing the potential across Cₚ. In accordance with certain exemplary embodiments of the present disclosure, a replica of the Cₚ compensation block/circuitry 220 can be used as part of CC to cancel out the latter contribution.

Lack of Rₛ compensation can lead to three primary deviations from the desired VC behavior. First, a step change in V command can result in a change in the membrane potential (Vₘ) with an exponential time constant determined by Rₛ Cₘ. Second, a current Iₚ flowing through Rₛ can cause Vₘ to deviate from V_{command} by Iₚ Rₛ. Lastly, signal current can be low-pass filtered with a time constant given by RₛCₘ. In a typical VC experiment in whole-cell configuration with Rₛ=25 MΩ and Cₘ=30 pF, this can set the 3-dB cutoff of this filter at 212 Hz. In accordance with certain exemplary embodiments of the present disclosure, 90% compensation of such Rₛ can increase the measurement bandwidth by 10× to 2.12 kHz.

To mitigate these deleterious effects, exemplary Rₛ compensation circuitry 230 of the present disclosure can be based on state estimator theory. For example, Vₘ can be estimated as V_{m,est} =Vₚ−Iₚ−R_{s,est} where Iₚ is the current flowing through Rₛ once Cₚ has been compensated, and R_{S,est} is the local estimate of Rₛ. Further, CMOS matching procedures can be exploited in order to feed an accurate copy of the current sensed by TIA 210 to the Rₛ compensation block/circuitry 230, which, e.g., can be the TIA itself with ten-bit programmable feedback resistance from 0 to 256 kΩ. Combined with the 1024× amplification in the current domain, this can facilitate the tuning of R_{s,est} up to 262 MΩ. The V_{m,est} thus generated can then be forced to equal an off-chip V command using negative feedback provided by an integrator implemented using a five-bit programmable transconductance block and a fixed 64-pF capacitor. Additional programmable low-pass filters can be included to assist in stabilize the overall loop. Achieving >75% Rₛ compensation is challenging and involves accurately measuring Iₚ at high bandwidths, typically exceeding 100 kHz. Further, the circuitry used for measuring Iₚ and generating V_{m,est} can be similar to that used in TIA 210, and can provide the same bandwidth benefits. Further, depending on the cell membrane capacitance, potentially 100% of Rₛ can be compensated.

FIG. 1E illustrates an exemplary diagram of the amplifier integrated circuit die in accordance with certain exemplary embodiments of the present disclosure. In particular, the figure illustrates a die photograph of the 3.225 mm×2.725 mm amplifier chip as manufactured in a 0.18 μm bulk CMOS process. The die can then be directly mounted on, and wirebonded to, a 1.4″×2″ custom-designed printed circuit board (PCB). The die can then be encapsulated with an epoxy in order to mechanically protect the wirebonds (see FIG. 1F). A connector can be included in order to connect to conventional pipette holders for use in patch experiments (see FIG. 1H). An aluminum enclosure 90 for the PCB can be provided in order to maintain compatibility with systems designed for commercial multi-clamp systems (see FIG. 1H). For example, the enclosure 60 can be mounted on a manipulator housed within a custom-designed microscope setup.

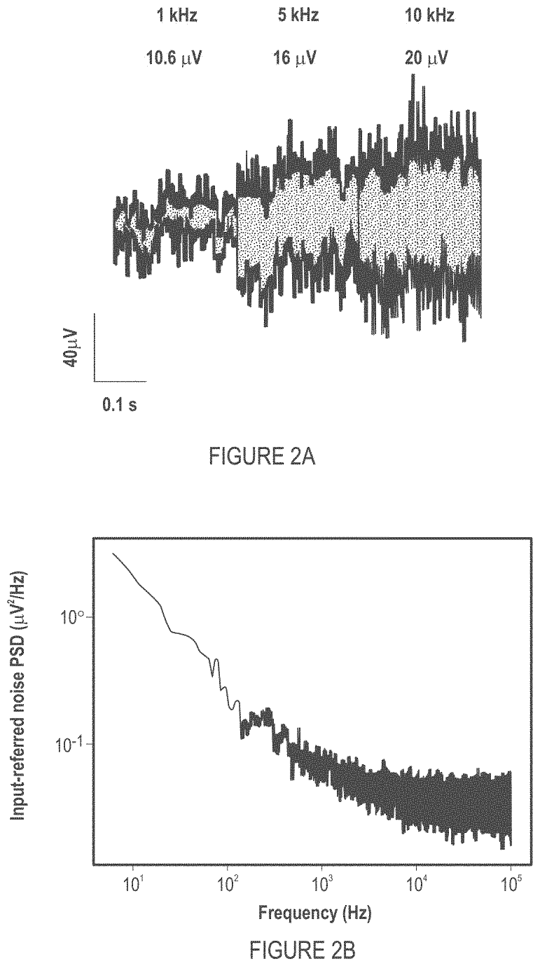

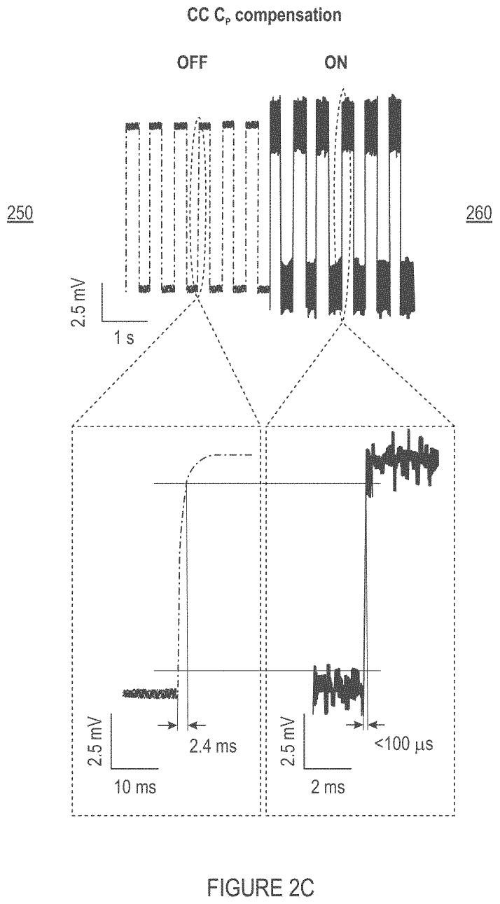

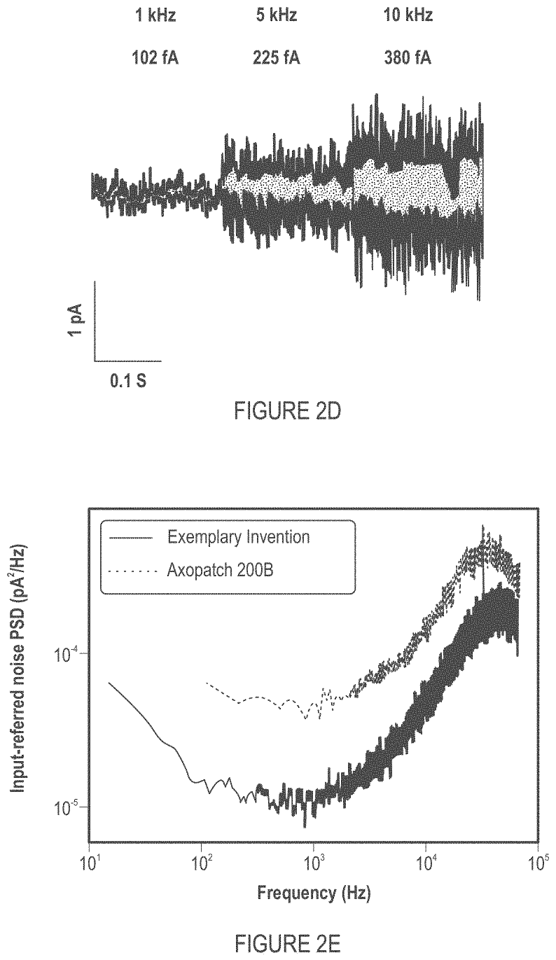

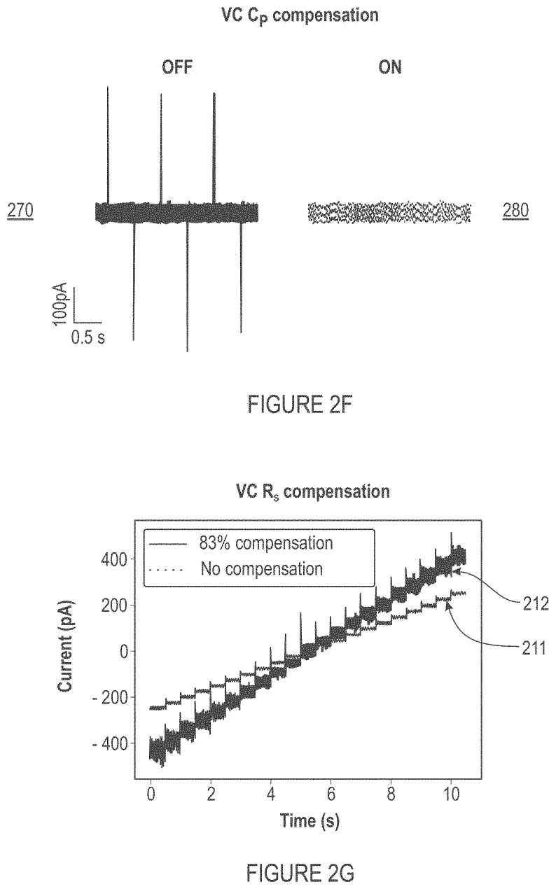

CC and VC function can be validated by using an electrical model cell (the example of which is shown in FIGS. 1B and 1D) comprising, e.g., Rₛ=100 MΩ, Rₘ=100 MΩ and Cₘ=20 pF for VC testing, and Rₘ=0 for CC testing. Cₚ can be determined by the parasitic trace capacitance to ground on the PCB. In this example, the noise performance of the CC voltage buffer 110 alone was determined first. FIG. 2A shows an example output time trace for a DC voltage source connected to the input, filtered to different bandwidths after acquisition, and FIG. 2B shows the corresponding input-referred voltage noise power spectral density (PSD) of the unfiltered time trace. In a 10-kHz bandwidth, the root-mean-square (RMS) value of the input-referred voltage noise is 20 μV_{RMS} and is dominated by the noise from on-PCB components. This can yield an acceptable signal-to-noise ratio (SNR) for recording extracellular action potentials and offers comparable performance to commercial instruments and previous integrated efforts.

FIG. 2C shows the voltage recorded by the buffer 110 (with and without capacitance compensation) for a 10-mVₚₚ-amplitude square wave at 2 Hz applied to V_{command} in order to inject a current square wave with a nominal amplitude of 100 pAₚₚ into Rₛ. The recorded amplitude of 10.1 mVₚₚ for the square wave indicates that the injected current is 10.1 mVₚₚ/100 MΩ=101 pAₚₚ. Without capacitance compensation 250, the injected current is filtered by the parallel combination of Rₛ and Cₚ and consequently, the measured voltage signal exhibits 10%-90% rise and fall times of 2.4 ms. With both current injection and capacitance compensation on simultaneously 260, rise and fall times of less than 100 μs can be observed, significantly faster than the case without Cₚ compensation. Cₚ compensation can operate substantially similarly to a voltage applied at Vₘ instead of V_{command}.

After characterizing the frequency response and linearity of TIA 210, the noise performance of TIA 210 can be determined. In VC mode, the current measured by TIA 210 can be provided by Iₚ=(V_{out,TIA}−Vₚ)/R_{f} (see FIG. 1D). With R_{f} set to ≈225 MΩ,

FIG. 2D plots the time trace of Iₚ for a constant externally applied Vₚ filtered to different bandwidths in software and FIG. 2E plots the corresponding PSD. FIG. 2E also shows the input-referred noise PSD for an Axopatch 200B (Molecular Devices) with R_{f} set to 500 MΩ, a commercially available system for ion channel recordings. TIA 210 according to certain exemplary embodiment of the present invention can generate only 225 fA_{RMS} of noise when filtered using a fourth-order 5 kHz Bessel filter. This is a factor of three better than the Axopatch 200B. Further, this is believed to be the lowest reported noise among known integrated multi-clamp amplifiers.

FIG. 2F shows the current recorded by TIA 210 (filtered to 10 kHz bandwidth) with (280) and without (270) Cₚ compensation for 1-Hz, 10-mVₚₚ steps in V_{command}. In accordance with certain exemplary embodiments of the present disclosure, prior to enabling Cₚ compensation, the current waveform can have large transient spikes at the onset of each step change in V_{command} due to the charging currents associated with changing the potential suddenly across the parasitic Cₚ. When tuned correctly, the transient charging currents can be removed completely from the recorded current. For example, the compensation circuitry can be tuned to remove 2 pF of parasitic capacitance.

After substantially eliminating the effect of Cₚ, the functionality of the Rₛ compensation circuitry can be tested with R_{f} set to 60 MΩ and the compensation tuned to reduce Rₛ by 83 MΩ. V command can be stepped from −50 mV to +50 mV in steps of 5 mV and the current recorded by TIA 210 can then be measured (see FIG. 2G). In accordance with certain exemplary embodiments of the present disclosure, in the absence of Rₛ compensation (e.g., graph 211), TIA 210 can apply this waveform across Rₛ+Rₘ resulting in the current varying from −250 pA to 250 pA in steps of 25 pA. Enabling Rₛ compensation increases the amplitude of the current step to 42.5 pA yielding an effective Rₛ of ≈17 MΩ, indicating that the Rₛ compensation circuit was successful in cancelling over 80% of the original Rₛ (e.g., graph 212 of FIG. 2G). Further, the spikes at the onset of each transition shown in FIG. 2G are more pronounced when Rₛ compensation is enabled. As the effective value of Rₛ decreases because of active compensation, the voltage applied across Cₘ more closely resembles the desired ideal step and results in larger charging currents. If the value of Cₘ is of the order of tens of fF, it is possible to completely compensate for Rₛ.

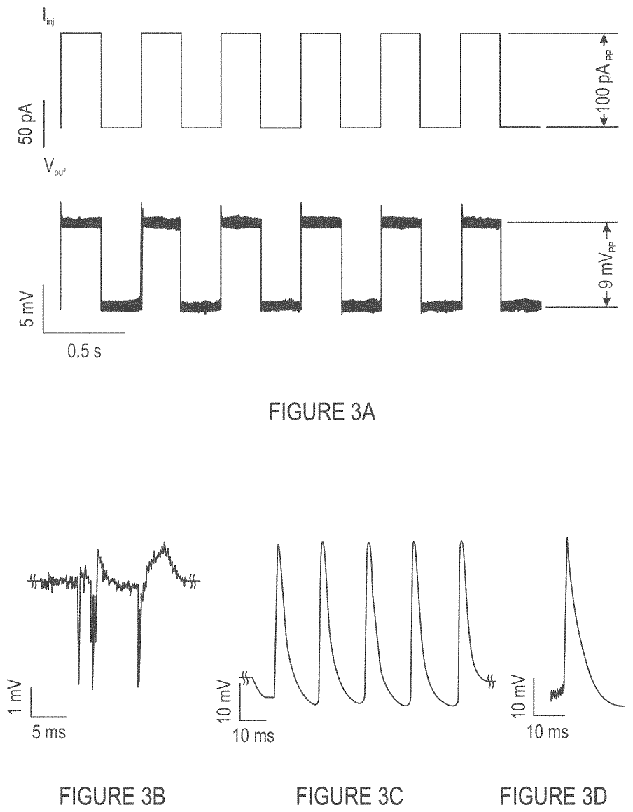

Further, in accordance with certain exemplary embodiments of the present disclosure, Rₛ and Cₚ of sharp microelectrodes can be characterized for use in CC mode by injecting a 2-Hz, 100-pAₚₚ signal into the electrode. FIG. 3A shows a voltage response obtained with a 100-nm high-impedance sharp microelectrode (e.g., 3 M KCl filling solution) immersed in a bath containing artificial cerebrospinal fluid (ACSF) with the Cₚ compensation circuitry 120 tuned to cancel 8 pF of parasitic capacitance. The response (filtered to 4 kHz) indicates a measured resistance of 90 MΩ with slight Cₚ overcompensation. This electrode can then be used to perform intra-as well as extracellular recordings from cortical layer-5 pyramidal neurons in acute slices. As depicted in FIGS. 3B and 3C, a resting membrane potential of −58 mV, and distinct extracellular (prior to cell entry) and intracellular neuronal action potentials with high SNR, millisecond time-scales, and ˜50-mV amplitudes can be observed. Further, as depicted in FIG. 3D, the exemplary device compares favorably to the MultiClamp 700B in terms of SNR, timescales, and signal fidelity.

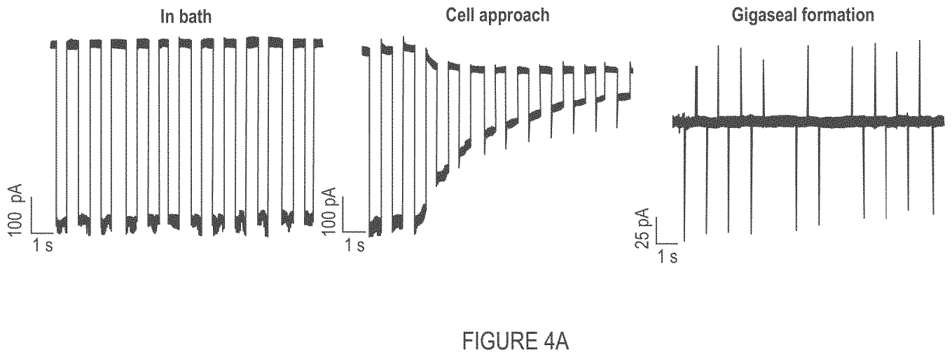

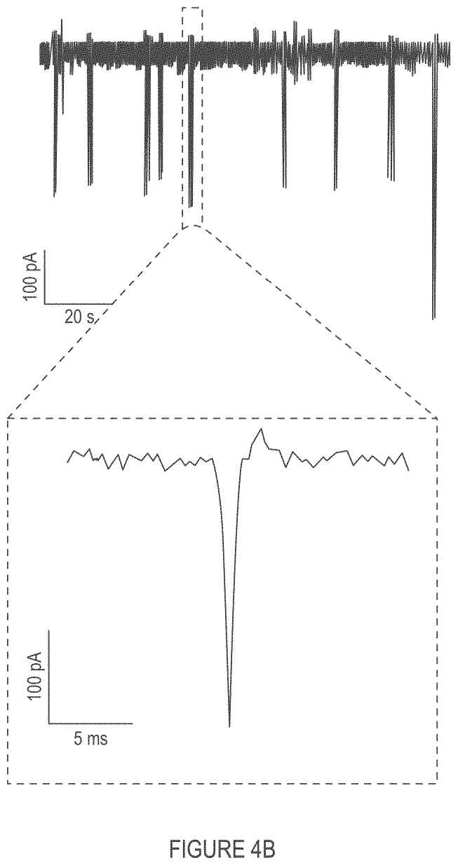

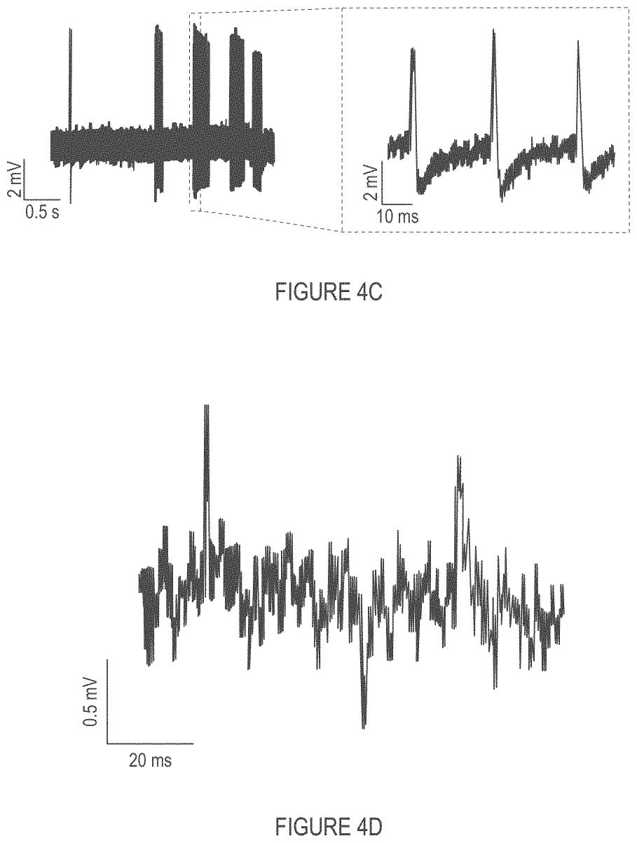

In VC mode, a periodic pulse with an amplitude of 5 mV and frequency of 1 Hz can be applied to determine the pipette 60's resistance prior to cell entry. The pipettes 60 can have resistances ranging from 7 to 14 MΩ. FIG. 4A shows the current recording (filtered to 1 kHz bandwidth) through one such pipette 60 in the bath, as it approaches the cell in 3-D cultures, and after formation of the giga-seal. In the cell-attached configuration (e.g., loose-seal; seal resistance can be approximately 150-200 Me), the pipette 60 was held at −70 mV and several spontaneous action potentials can be observed, as shown in FIG. 4B. In another experiment, spontaneous action potentials in CC were observed as well (see FIG. 4C, filtered to 10-kHz bandwidth). In accordance with certain exemplary embodiments of the present disclosure, current was injected to maintain approximately −50 mV in the pipette. Further, the signals were characterized by high SNR biphasic waveforms and amplitudes of several mV indicating that these were tightly-coupled extracellular action potentials due to incomplete rupture. Upon rupturing the membrane further, and when filtered to 2-kHz bandwidth, excitatory and inhibitory postsynaptic potentials were also observed (see FIG. 4D).

In accordance with certain exemplary embodiments of the present disclosure, the capacitance compensation circuit/block 120 can utilize positive feedback. The filtered membrane voltage at the pipette (Vₚ) can be sensed and buffered through the voltage buffer 110 as V_{buf}. The buffered voltage can then be multiplied by a scaling factor A with magnitude between 1 and 2. Lower values of A can help reduce the noise injected by the capacitance compensation circuitry. AV_{buf} can then be applied to one terminal of the injection capacitor with the other terminal connected to the input of the voltage buffer and the pipette establishing a potential difference of (A−1)Vₚ, across the capacitor if V_{buf} ≈Vₚ. The stability of the capacitance compensation loop can worsen as the difference between the two values decreases.

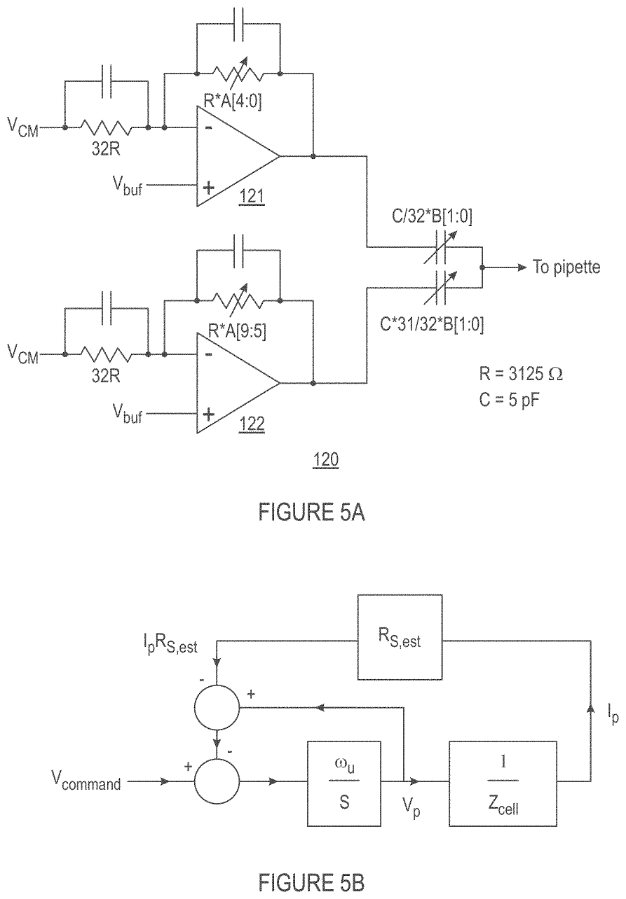

FIG. 5A shows the block diagram of the on-chip implementation of the capacitance compensation circuitry/block 120. In accordance with certain exemplary embodiments of the present disclosure, A is a ten-bit digitally programmable value. Preferably, each of the op-amps in the non-inverting amplifier configuration, e.g., op-amps 121, 122, can provide a gain to V_{buf} between 1 and 2 and proportional to the value of the appropriate bits of A. The upper path, e.g., op-amp 121, can be considered to provide the “fine” control over the amount of capacitance compensation while the lower path, e.g., op-amp 122, can provide the “coarse” control. Splitting the amplification into two blocks can increase the overall power consumption but can decrease area and layout complexity and can reduce the effect of parasitic capacitances in the programmable resistors.

In accordance with certain exemplary embodiments of the present disclosure, for a current Iₚ flowing through the pipette 60, the membrane potential can be expressed as

Vₘ=Vₚ−IₚRₛ

where Vₚ is the voltage applied to the positive terminal of TIA 210 and appears on the pipette 60 through the clamping action of TIA 210. Further, in the ideal case where Rₛ=0, the membrane voltage can be exactly equal to Vₚ.

FIG. 5B illustrates an exemplary block diagram of a feedback loop implemented in the resistance compensation circuit 230. A voltage Vₚ can be applied across the pipette 60 and the cell can generate a proportional current Iₚ. This current can be passed through a local estimate of the pipette's Rₛ (R_{s,est}) and subtracted from Vₚ to generate a local estimate of the membrane voltage as

V_{m,est}=Vₚ−IₚR_{s,est}

This voltage can then be compared to V command, with the error being fed to an integrator. In a negative feedback loop, the action of the integrator can drive its input to zero implying V_{command}=V_{m,est}. For α=R_{s,est}/Rₛ, Vₘ=Vₘₑₛₜ when α=1 indicating full Rₛ compensation if the resultant feedback loop is stable.

If the loop is broken at the input of the integrator, the loop gain can be written (excluding the negative sign) as

$$L ( s ) = \frac{{ω}_{u}}{S} ( 1 - \frac{{R}_{s , est}}{{Z}_{cell}} )$$

where, in whole-cell configuration neglecting the effect of Rₘ, Z_{cell} can be expressed as

$${Z}_{cell} = \frac{( {R}_{s} + \frac{1}{{sC}_{m}} ) \frac{1}{{sC}_{p}}}{{R}_{s} + \frac{1}{{sC}_{m}} + \frac{1}{{sC}_{p}}}$$

where Cₚ represents the amount of uncompensated capacitance and can be reduced to <100 fF levels. Two cases of particular interest are described below

$${Z}_{cell} = \{ \begin{matrix}\frac{1}{{sC}_{m}} \frac{1 + {s τ}_{m}}{1 + {s τ}_{p}} & if {C}_{m} >> {C}_{p} \\ \frac{1}{{sC}_{m}} \frac{1 + {s τ}_{m}}{2 + {s τ}_{m}} & if {C}_{m} \approx {C}_{p}\end{matrix}$$

where τₘ=RₛCₘ and τₚ=RₛCₚ. In the first case, the loop gain can then be rewritten as

$$L ( s ) = \frac{{ω}_{u}}{s} ( 1 - \frac{{α s τ}_{m} ( 1 + {s τ}_{p} )}{1 + {s τ}_{m}} ) = \frac{{ω}_{u}}{s} ( \frac{1 + ( 1 - α ) {s τ}_{m} - {α s}^{2} {τ}_{m} {τ}_{p}}{1 + {s τ}_{m}} )$$

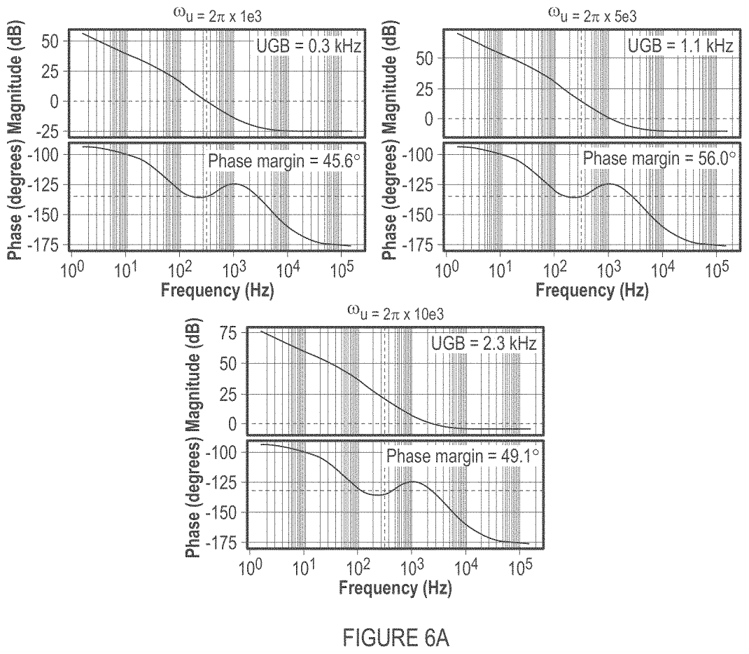

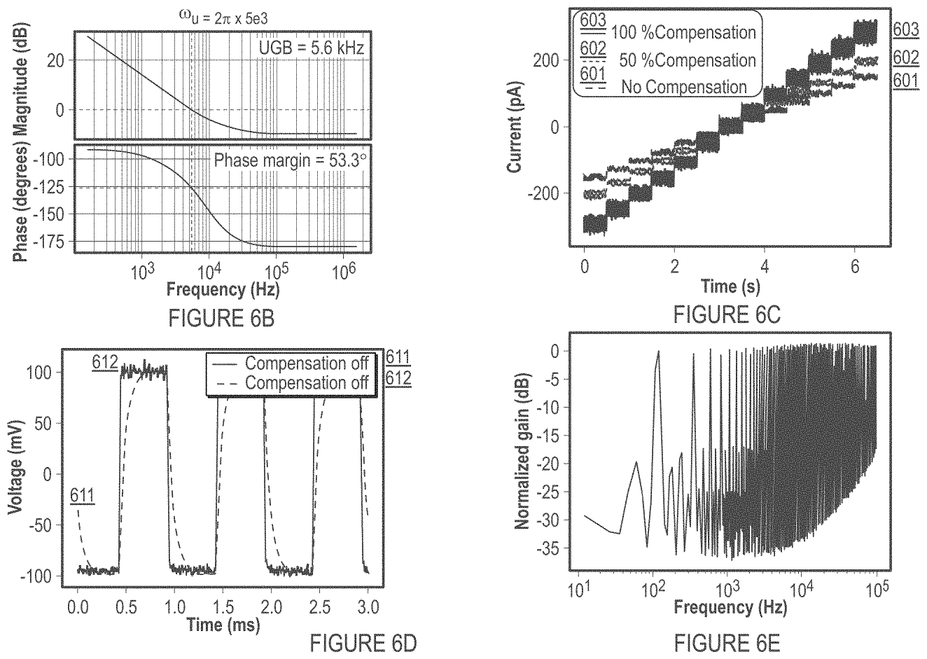

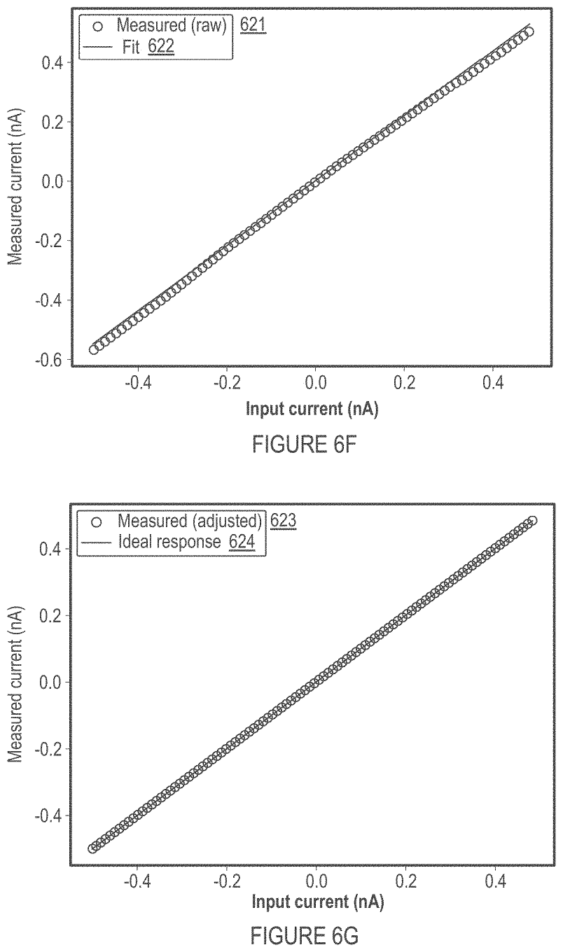

FIG. 6A shows an exemplary Bode plot for this equation with Rₛ=100 MΩ, ₘ=20 pF, Cₚ=100 fF, and α=0.83 for various values of ωᵤ. The system can have one pole at zero and one at −1/τₘ. So long as α<1, the left-half plane zero appears before the right-half plane zero and aids in stability if ωᵤ is large enough such that the left-half plane zero appears before the unity-gain crossing frequency. If α=1, the left-half plane and right-half plane both appear at the same frequency, providing no improvement in phase margin but requiring additional poles for returning to the single pole roll-off condition (and thereby worsening phase margin).

In the case when both Cₘ and Cₚ are small and <100 fF, the loop gain can be written as

$$L ( s ) = - \frac{{ω}_{u}}{s} ( 1 - \frac{{α s τ}_{m} ( 2 + {s τ}_{m} )}{1 + {s τ}_{m}} ) = - \frac{{ω}_{u}}{s} ( \frac{1 + ( 1 - 2 α ) {s τ}_{m} - {α s}^{2} {τ}_{m}^{2}}{1 + {s τ}_{m}} )$$

where Rₛ=100 MΩ, Cₘ=100 fF, Cₚ=100, and α=1. FIG. 6B plots the Bode response for such a system. The zeros and the pole can be moved beyond the loop bandwidth since τₘ=10 μs in this example. For this loop gain, stability at 100% compensation is possible at bandwidths greater than 5 kHz.

FIG. 6C shows an exemplary plot of the current recorded by an exemplary TIA 210 when connected to an electrical model cell with Rₛ=Rₘ=100 MΩ. Cₘ can be determined by trace parasitic capacitances and can be estimated to be on the order of 10 fF. Cₚ can be substantially reduced by using the associated compensation circuitry 220. V_{command} can then be stepped from −30 mV to +30 mV in steps of 5 mV. When Rₛ compensation is disabled (e.g., graph 601), the changes in V_{command} can then yield currents that change from −150 pA to +150 pA in steps of 50 pA, since the voltage appears across Rₛ+Rₘ. With Rₛ compensation enabled and tuned to cancel out ≈50 MΩ, the current can vary from −200 pA to +200 pA (Rₛ+Rₘ=150 MΩ) (e.g., graph 602). Finally, with Rₛ compensation set to cancel approximately 100 MΩ, V_{command} can be applied almost entirely across Rₘ and Cₘ, resulting in a further increase in the current amplitudes with the current varying from nearly −300 pA to +300 pA (e.g., graph 603).

In accordance with certain exemplary embodiments of the present disclosure, the functionality of the CC Cₚ compensation circuitry/block 120 can be tested for inputs applied at Vₘ. For example, a function generator can be used to inject a square voltage wave with an amplitude of 200 mVₚₚ, a DC offset of 1.65 V, and a frequency of 1 kHz at Vₘ. Further, Rₛ=20 MΩ and Cₚ can be determined by parasitic capacitance on the trace connected to the input of the CC. In the absence of capacitance compensation, the waveform as shown in FIG. 6D can be observed, with a 10%-90% rise time of approximately 350 μs (e.g., graph 611). FIG. 6D also shows the output when Cₚ compensation is turned on and tuned to approximately 3.5 pF (e.g., graph 612). The rise time recorded in this case is approximately 20 μs and approaches the limit of the recording bandwidth determined by the cutoff frequency of the anti-aliasing filter.

Further, in accordance with certain exemplary embodiments of the present disclosure, the frequency response of TIA 210 can be determined in two parts. First, the DC gain can be determined by injecting a known current into TIA 210 and recording the corresponding output voltage. The response as a function of the input frequency can determined by coupling a square wave of voltage into TIA 210 input through a small capacitor. The small capacitor can be realized by holding the wire connected to the square voltage wave near TIA 210 input. The exact value of the capacitance can be hard to determine but is not necessary to be known. This setup can inject an impulse train of current into TIA 210 with alternating positive and negative impulses. By ensuring that the impulse amplitude is small enough to not saturate TIA 210 and by choosing an appropriate square wave frequency, the Fourier transform of such a time-domain signal can be an impulse train in the frequency domain consisting of odd harmonics of the injected frequency. The amplitudes of these frequency domain impulses can then yield the AC gains at those frequencies. FIG. 6E illustrates exemplary graphs of this AC gain of an exemplary TIA 210 with gain set to 225 MΩ and normalized to the value of the gain at the fundamental frequency.

FIG. 6F shows exemplary graphs of the linearity of the measured current as the input current is swept from −500 pA to +500 pA in steps of 10 pA with R_{f}=120 MΩ (e.g., graph 621). The current can be injected through Rₛ+Rₘ=20+100=120 MΩ by stepping the voltage applied at the other end of Rₘ. A linear least-squares fit can then be generated from the measured data (e.g., graph 622). Non-idealities in the op amps and mismatches in the threshold voltages of the subthreshold transistors can cause systematic deviation from the linear fit where positive and negative currents have slightly different gains. FIG. 6G shows the resultant input-output characteristic after dividing all the positive currents by 1.042 and negative currents by 1.133. The line shown in this figure represents the ideal y=x transfer characteristic (e.g., graph 624) and the adjusted data (e.g., graph 623) in this example are remarkably similar.

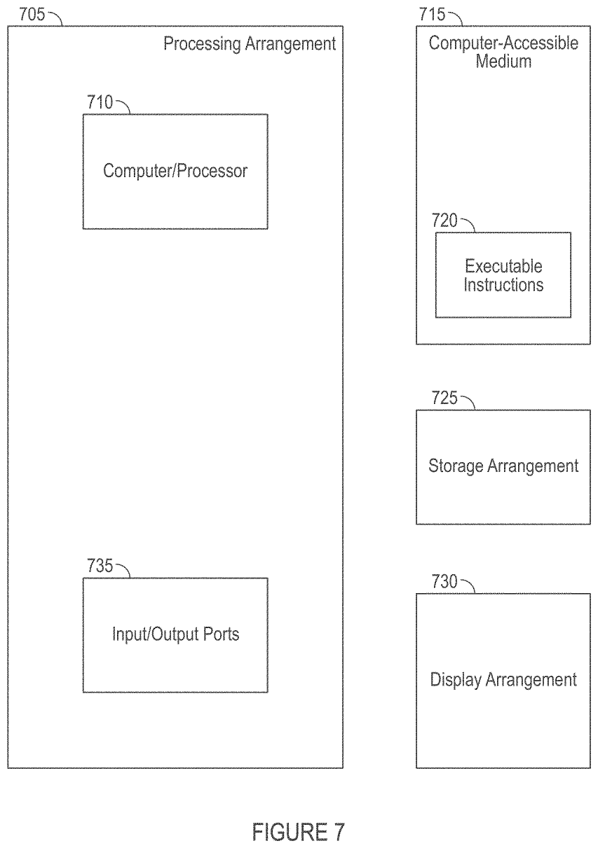

FIG. 7 shows a block diagram of an exemplary embodiment of a system according to the present disclosure. For example, exemplary procedures in accordance with the present disclosure described herein can be performed by a processing arrangement and/or a computing arrangement (e.g., computer hardware arrangement) 705. Such processing/computing arrangement 705 can be, for example entirely or a part of, or include, but not limited to, a computer/processor 710 that can include, for example one or more microprocessors, and use instructions stored on a computer-accessible medium (e.g., RAM, ROM, hard drive, or other storage device).

As shown in FIG. 7, for example a computer-accessible medium 715 (e.g., as described herein above, a storage device such as a hard disk, floppy disk, memory stick, CD-ROM, RAM, ROM, etc., or a collection thereof) can be provided (e.g., in communication with the processing arrangement 705). The computer-accessible medium 715 can contain executable instructions 720 thereon. In addition or alternatively, a storage arrangement 725 can be provided separately from the computer-accessible medium 715, which can provide the instructions to the processing arrangement 705 so as to configure the processing arrangement to execute certain exemplary procedures, processes, and methods, as described herein above, for example.

Further, the exemplary processing arrangement 705 can be provided with or include an input/output ports 735, which can include, for example a wired network, a wireless network, the intemet, an intranet, a data collection probe, a sensor, etc. As shown in FIG. 7, the exemplary processing arrangement 705 can be in communication with an exemplary display arrangement 730, which, according to certain exemplary embodiments of the present disclosure, can be a touch-screen configured for inputting information to the processing arrangement in addition to outputting information from the processing arrangement, for example. Further, the exemplary display arrangement 730 and/or a storage arrangement 725 can be used to display and/or store data in a user-accessible format and/or user-readable format.

The foregoing merely illustrates the principles of the disclosure. Various modifications and alterations to the described embodiments will be apparent to those skilled in the art in view of the teachings herein. It will thus be appreciated that those skilled in the art will be able to devise numerous systems, arrangements, and procedures which, although not explicitly shown or described herein, embody the principles of the disclosure and can be thus within the spirit and scope of the disclosure. Various different exemplary embodiments can be used together with one another, as well as interchangeably therewith, as should be understood by those having ordinary skill in the art. In addition, certain terms used in the present disclosure, including the specification and drawings, can be used synonymously in certain instances, including, but not limited to, for example, data and information. It should be understood that, while these words, and/or other words that can be synonymous to one another, can be used synonymously herein, that there can be instances when such words can be intended to not be used synonymously. Further, to the extent that the prior art knowledge has not been explicitly incorporated by reference herein above, it is explicitly incorporated herein in its entirety. All publications referenced are incorporated herein by reference in their entireties.

### EXEMPLARY REFERENCES

The following references are hereby incorporated by reference in their entireties:
1. Sakmann, B. & Neher, E. Single Channel Recording—Second Edition. (Springer Science & Business Media, 1995);
2. Purves, R. D. Microelectrode Methods for Intracellular Recording and Ionophoresis. (Academic Press, 1981);
3. Inc., A. I. Axopatch 200B Patch Clamp—Theory and Operation. (1999). Available at: https://www.autom8.com/wp-content/uploads/2016/07/Axopatch-200B.pdf;
4. Molecular Devices. MultiClamp 700B Amplifier. Available at: https://www.moleculardevices.com/systems/conventional-patch-clamp/multiclamp-700b-microelectrode-amplifier. (Accessed: 10th September 2017); p0 5. Laiwalla, F., Klemic, K. G., Sigworth, F. J. & Culurciello, E. An integrated patch-clamp amplifier in silicon-on-sapphire CMOS. IEEE Trans. Circuits Syst. I Regul. Pap. 53, 2364-2370 (2006);
6. Weerakoon, P. et al. Patch-clamp amplifiers on a chip. J. Neurosci. Methods 192, 187-192 (2010);
7. Kim, J., Maitra, R., Pedrotti, K. D. & Dunbar, W. B. A patch-clamp ASIC for nanopore-based DNA analysis. IEEE Trans. Biomed. Circuits Syst. 7, 285-295 (2013);
8. Li, H. et al. Ultracompact Microwatt CMOS Current Readout with Picoampere Noise and Kilohertz Bandwidth for Biosensor Arrays. IEEE Trans. Biomed. Circuits Syst. 12, 35-46 (2018);
9. Dai, S. & Rosenstein, J. K. A 15-V Bidirectional Current Clamp Circuit for Integrated Patch Clamp Electrophysiology. IEEE Trans. Circuits Syst. II Express Briefs 64, 1287-1291 (2017);
10. Goldstein, B., Choe, K., Sigworth, F. J. & Culurciello, E. A four-channel integrated patch-clamp amplifier with current-clamp capability. in Midwest Symposium on Circuits and Systems 1-4 (2011);
11. Harrison, R. R. et al. Microchip amplifier for in vitro, in vivo, and automated whole-cell patch-clamp recording. J. Neurophysiol. 113, 1275-1282 (2014);
12. Sigworth, F. J. Design of the EPC-9, a computer-controlled patch-clamp amplifier. 1. Hardware. J. Neurosci. Methods 56, 195-202 (1995);
13. Cole, K. S. Membranes, ions and impulses: A chapter of classical biophysics. (Univ. of California Press, 1972);
14. Larkum, M. E., Nevian, T., Sandier, M., Polsky, A. & Schiller, J. Synaptic integration in tuft dendrites of layer 5 pyramidal neurons: A new unifying principle. Science. 325, 756-760 (2009);
15. Jayant, K. et al. Targeted intracellular voltage recordings from dendritic spines using quantum-dot-coated nanopipettes. Nat. Nanotechnol. 12, 335-342 (2017);
16. English, D. F. et al. Excitation and Inhibition Compete to Control Spiking during Hippocampal Ripples: Intracellular Study in Behaving Mice. J. Neurosci. 34, 16509-16517 (2014);
17. Beaulieu-Laroche, L. & Harnett, M. T. Dendritic Spines Prevent Synaptic Voltage Clamp. Neuron 97, 75-82 (2018);
18. Razavi, B. Design of Analog CMOS Integrated Circuits. (McGraw Hill, 2016);
19. Ferrari, G., Gozzini, F., Molari, A. & Sampietro, M. Transimpedance amplifier for high sensitivity current measurements on nanodevices. in IEEE Journal of Solid-State Circuits 44, 1609-1616 (2009);
20. Ferrari, G., Farina, M., Guagliardo, F., Carminati, M. & Sampietro, M. Ultra-low-noise CMOS current preamplifier from DC to 1 MHz. Electron. Lett. 45, 1278-1280 (2009);
21. Sherman, A. J., Shrier, A. & Cooper, E. Series Resistance Compensation for Whole-Cell Patch-Clamp Studies Using a Membrane State Estimator. Biophys. 1 77, 2590-2601 (1999);
22. Long, M. A., Jin, D. Z. & Fee, M. S. Support for a synaptic chain model of neuronal sequence generation. Nature 468, 394-399 (2010);
23. Perkins, K. L. Cell-attached voltage-clamp and current-clamp recording and stimulation techniques in brain slices. J Neurosci. Methods 154, 1-18 (2006);
24. Fromherz, P. Extracellular recording with transistors and the distribution of ionic conductances in a cell membrane. Eur. Biophys. 1 28, 254-258 (1999);
25. Beaudoin III, G. M. J. et al. Culturing pyramidal neurons from the early postnatal mouse hippocampus and cortex. Nat. Protoc. 7, 1741-1754 (2012); and
26. Dingle, Y.-T. L. et al. Three-Dimensional Neural Spheroid Culture: An In Vitro Model for Cortical Studies. Tissue Eng. Part C Methods 21, 1274-1283 (2015).

1. An integrated electrophysiology amplifying system, comprising:
a pipette interface configured to receive a device which is a pipette or a sharp microelectrode; and
an integrated circuit comprising (i) an amplifier coupled to the pipette interface and configured to control a current through the pipette or record a cell membrane voltage, and (ii) at least one compensation circuit using negative feedback,
wherein the integrated circuit and the pipette interface are physically integrated within a common housing.

2. The system of claim 1, wherein the amplifier is further configured to control the cell membrane voltage or record a trans-membrane current.

3. The system of claim 2, wherein the amplifier includes (i) a current-clamp module to control the current through the pipette, and (ii) a voltage-clamp module to control the cell membrane voltage.

4. The system of claim 3, wherein the current-clamp and voltage-clamp modules share an input.

5. The system of claim 1, wherein at least one compensation circuit compensates a series resistance associated with the pipette.

6. The system of claim 5, wherein the at least one compensation circuit compensates for the series resistance over a range greater than 100 MΩ.

7. The system of claim 6, wherein the amount of series resistance compensated is programmed via a digital interface.

8. The system of claim 1, wherein at least one compensation circuit compensates for a capacitance associated with the pipette.

9. The system of claim 8, wherein the at least one compensation circuit compensates for the capacitance associated with the pipette over a range greater than 10 pF.

10. The system of claim 9, wherein an amount of the capacitance compensated by the at least one compensation circuit is programmed via a digital interface.

11. An integrated electrophysiology amplifying system, comprising:
a pipette interface for receiving a pipette or a sharp microelectrode; and
an integrated circuit comprising (i) an amplifier coupled to the pipette interface and configured to control a cell membrane voltage or record a trans-membrane current, and (ii) at least one compensation circuit using negative feedback,
wherein the integrated circuit and the pipette interface are physically integrated within a common housing.

12. The system of claim 11, wherein the amplifier is further configured to control the cell membrane current or record a trans-membrane voltage.

13. The system of claim 12, wherein the amplifier includes (i) a current-clamp module to control the voltage through the pipette, and (ii) a voltage-clamp module to control the cell membrane voltage.

14. The system of claim 13, wherein the current-clamp module and the voltage-clamp module share an input.

15. The system of claim 11, wherein at least one compensation circuit compensates a series resistance associated with the pipette.

16. The system of claim 15, wherein the at least one compensation circuit compensates for the series resistance over a range greater than 100 MΩ.

17. The system of claim 16, wherein the amount of series resistance compensated by the at least one compensation circuit is programmed via a digital interface.

18. The system of claim 11, wherein at least one compensation circuit compensates for a capacitance associated with the pipette.

19. The system of claim 18, wherein at least one compensation circuit compensates for the capacitance associated with the pipette over a range greater than 10 pF.

20. The system of claim 19, wherein an amount of the capacitance compensated by the at least one compensation circuit is programmed via a digital interface.

21. A method for using or providing an integrated electrophysiology amplifying system, the method comprising:
facilitating a receipt of a device which is a pipette or a sharp microelectrode using a pipette interface of the system; and
controlling at least one of (i) a cell membrane, or (ii) a current through the device using an amplifier of an integrated circuit which is coupled to the pipette interface, wherein the integrated circuit comprises at least one compensation circuit using negative feedback,
wherein the integrated circuit and the pipette interface are physically integrated within a common housing.

22. A method for using or providing an integrated electrophysiology amplifying system, the method comprising:
facilitating a receipt of a pipette or a sharp microelectrode using a pipette interface of the system; and
recording at least one of a cell membrane voltage or a trans-membrane current using an amplifier coupled to the pipette interface, wherein the integrated circuit comprises at least one compensation circuit using negative feedback,
wherein the integrated circuit and the pipette interface are physically integrated within a common housing.

23 and 24. (canceled)
